(19) United States
(12) Patent Application Publication
(10) Pub. No.: US 2021/0355082 A1
(43) Pub. Date: Nov. 18, 2021

(54) METHOD FOR PRODUCING SALT

(71) Applicant: FUJIFILM Corporation, Tokyo (JP)

(72) Inventors: Ryo MINAMIKAWA, Haibara-gu (JP); Kazunari YAGI, Haibara-gu, Shizuoka (JP); Akiyoshi GOTO, Haibara-gu (JP)

(73) Assignee: FUJIFILM Corporation, Tokyo (JP)

(21) Appl. No.: 17/391,301

(22) Filed: Aug. 2, 2021

Related U.S. Application Data
(63) Continuation of application No. PCT/JP2020/007539, filed on Feb. 25, 2020.

(30) Foreign Application Priority Data
Feb. 26, 2019 (JP) 2019-033212

(51) Int. Cl. C07C 381/12; G03F 7/004

(52) U.S. Cl. CPC C07C 381/12; C07C 51/412; G03F 7/0045

(57) ABSTRACT

According to the present invention, there is provided a method of producing a salt, including reacting M⁺X⁻ with YH to generate XH and M⁺Y⁻ and subsequently removing the generated XH to obtain the M⁺Y⁻.

In the method of producing a salt, M⁺X⁻ is a salt of a cation represented by M⁺ and an anion represented by X⁻, M⁺Y⁻ is a salt of the cation represented by M⁺ and an anion represented by Y⁻, XH is a conjugate acid of X⁻, YH is a conjugate acid of Y⁻, M⁺Y⁻ is a compound that generates an acid upon irradiation with an active ray or a radioactive ray, a pKa of XH is larger than a pKa of YH, and a ClogP value of XH is larger than 2.

### CROSS REFERENCE TO RELATED APPLICATIONS

This is a continuation of International Application No. PCT/JP2020/007539 filed on Feb. 25, 2020 and claims priority from Japanese Patent Application No. JP2019-033212 filed on Feb. 26, 2019, the entire content of which is incorporated herein by reference.

### BACKGROUND OF THE INVENTION

### 1. Field of the Invention

The present invention relates to a method of producing a salt. More specifically, the present invention relates to a method of producing a salt, which is a compound that generates an acid upon irradiation with an active ray or a radioactive ray.

### 2. Description of the Related Art

A salt having a sulfonium ion or an iodonium ion as a cation moiety is widely used as a compound (a photo-acid generator) that generates an acid upon irradiation with an active ray or a radioactive ray, for example, in a photolithography process in the field of semiconductor manufacturing.

In the related art, a salt containing a hydrophilic anion moiety such as a hydrogen carbonate ion has been produced, for example, by ion-exchanging a halogenated onium salt using an anion exchange resin.

For example, WO2015/019983A discloses a method of producing a sulfonium salt compound, in which a triarylsulfonium halide is ion-exchanged using an ion exchange resin, and the obtained hydrogen carbonate sulfonium salt is used as an intermediate.

Further, JP2017-3927A discloses a resist composition containing a hydrogen carbonate sulfonium salt.

### SUMMARY OF THE INVENTION

In the production of a photo-acid generator that is used in a resist composition, it is common to perform liquid separation purification (washing with water) for reducing metal impurities after synthesizing the target product by the ion exchange.

However, for example, a salt containing a hydrophilic anion such as acetate ion and a hydrogen carbonate ion is highly water-soluble and metal impurities are also present in the aqueous layer, and thus it is impossible to remove metal impurities by liquid separation purification (washing with water). As a result, it is inevitable that the photo-acid generator is contaminated by metal impurities and thus the quality control has been difficult in terms of the metal content. In particular, in the field of semiconductor manufacturing, the standard of metal content is strict, and it is required to reduce the content of metal impurities in the photo-acid generator.

An object of the present invention is to provide a method of producing a photo-acid generator having a low content of metal impurities.

The inventors of the present invention have conducted intensive studies to solve the above problems and as a result, have found that the above problems can be solved by the following configurations. That is, the present invention is as follows.

[1] A method of producing a salt, comprising:

reacting M⁺X⁻ with YH to generate XH and M⁺Y⁻, and subsequently removing the generated XH to obtain the M⁺Y⁻.

in which the M⁺X⁻ is a salt of a cation represented by M⁺ and an anion represented by X⁻,

the M⁺Y⁻ is a salt of the cation represented by M⁺ and an anion represented by the XH is a conjugate acid of X⁻,

the YH is a conjugate acid of X⁻,

the M⁺Y⁻ is a compound that generates an acid upon irradiation with an active ray or a radioactive ray,

a pKa of the XH is larger than a pKa of the YH, and

a ClogP value of the XH is larger than 2.

[2] The method of producing a salt according to [1], in which a ClogP value of the YH is smaller than the ClogP value of the XH.

[3] The method of producing a salt according to [1] or [2], in which the reaction of the M⁺Y⁻ with the YH is carried out in a reaction solvent at −78° C. or higher and 100° C. or lower.

[4] The method of producing a salt according to [3], in which the reaction solvent is an ether-based solvent, an ester-based solvent, a ketone-based solvent, a nitrile-based solvent, an alcohol-based solvent, or a fluorine-based solvent.

[5] The method of producing a salt according to any one of [1] to [4] in which a crystal containing the M⁺Y⁻ or an oily product containing the M⁺Y⁻ is obtained by the reaction of the M⁺X⁻ with the YH, and the crystal is washed with a washing solvent or the oily product is distilled off under reduced pressure to remove the XH contained in the crystal or the oily product.

[6] The method of producing a salt according to [5], in which the washing solvent is an ether-based solvent, an ester-based solvent, a ketone-based solvent, a nitrile-based solvent, an alcohol-based solvent, or a fluorine-based solvent.

[7] The method of producing a salt according to any one of [1] to [6], in which the M⁺ is a sulfonium ion or an iodonium ion.

[8] The method of producing a salt according to any one of [1] to [7], in which the X⁻ and the Y⁻ are each independently an anion represented by any one of General Formulae (1) to (6).

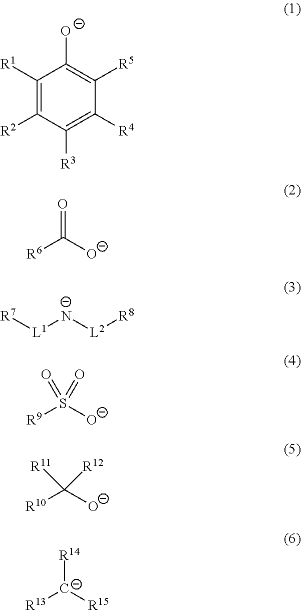

In General Formula (1), R¹ to R⁵ each independently represent a hydrogen atom, a halogen atom, a hydroxy group, or a monovalent organic group. Here, at least two of R¹, . . . , or R⁵ may be bonded to form a ring structure.

In General Formula (2), R⁶ represents a hydrogen atom, a halogen atom, a hydroxy group, or a monovalent organic group.

In General Formula (3), R⁷ and R⁸ each independently represent a hydrogen atom, a halogen atom, a hydroxy group, or a monovalent organic group. Here, R⁷ and R⁸ may be bonded to form a ring structure. L¹ represents —SO₂—, —C(═O)—, or a single bond, and L² represents —SO₂—, or —C(═O)—.

In General Formula (4), R⁹ represents a hydrogen atom, a halogen atom, a hydroxy group, or a monovalent organic group.

In General Formula (5), R¹⁰ to R¹² each independently represent a hydrogen atom, a halogen atom, a hydroxy group, or a monovalent organic group.

In General Formula (6), R′³ to R¹⁵ each independently represent —SO₂—R¹⁶, —C(═O)—R¹⁶, or a cyano group. Here, R¹⁶ represents a hydrogen atom, a halogen atom, a hydroxy group, or a monovalent organic group.

[9] The method of producing a salt according to [8], in which the X⁻ is an anion represented by General Formula (1) or (3).

[10] The method of producing a salt according to [8] or [9], in which the X⁻ is represented by General Formula (I), and at least one of R¹, . . . , or R⁵ in General Formula (I) represents a monovalent organic group.

[11] The method of producing a salt according to any one of [8] to [10], in which the Y⁻ is any one of:

an anion represented by General Formula (1), where R¹ to R⁵ in General Formula (1) represent a halogen atom,

an anion represented by General Formula (2), where R⁶ in General Formula (2) represents a hydroxy group or a monovalent organic group,

an anion represented by General Formula (3), where R⁷ and R⁸ in General Formula (3) represent a monovalent organic group,

an anion represented by General Formula (4), where R⁹ in General Formula (4) represents a monovalent organic group,

an anion represented by General Formula (5), where R¹⁰ to R¹² in General Formula (5) each independently represent a monovalent organic group, or

an anion represented by General Formula (6), where R¹³ to R¹⁵ in General Formula (6) each independently represent —SO₂—R¹⁶, and R¹⁶ represents a monovalent organic group.

[12] The method of producing a salt according to any one of [1] to [11], further comprising:

reacting M⁺G⁻ with Q⁺X⁻ to generate the M⁺X⁻ and Q⁺G⁻, and subsequently, removing the generated Q⁺G⁻ to obtain M⁺X⁻,
where the G⁻ is a halogen ion, and the Q⁺ is an alkali metal ion or an ammonium ion.

[13] The method of producing a salt according to [12], in which the reaction of the M⁺G⁻ with the Q⁺X⁻ is carried out in a presence of an organic solvent and water, and an obtained organic layer is washed with water to obtain M⁺X⁻.

[14] The method of producing a salt according to [12] or [13], in which the G⁻ is a bromine ion or a chlorine ion.

According to the present invention, it is to provide a method of producing a photo-acid generator having a low content of metal impurities.

### DESCRIPTION OF THE PREFERRED EMBODIMENTS

In the following description, the present invention will be described in more detail.

The explanation of the constituent elements described below may be based on representative embodiments of the invention; however, the present invention is not intended to be limited to those embodiments.

In the present specification, the term “active ray” or “radioactive ray” means, for example, the bright line spectrum of a mercury lamp, a far ultraviolet ray represented by an excimer laser, an extreme ultraviolet ray (EUV), an X-ray, an electron beam (EB), and the like. In the present specification, the term “light” means an active ray or a radioactive ray.

In the present specification, “to” is used to mean that numerical values described before and after “to” are included as a lower limit value and an upper limit value, respectively.

In the present specification, the pKa (the acid dissociation constant pKa) represents an acid dissociation constant pKa in an aqueous solution and is defined in, for example, Handbook of Chemistry (II) (4th Revised Edition, 1993, edited by the Chemical Society of Japan, Maruzen Publishing Co., Ltd.). The lower the value of the acid dissociation constant pKa, the larger the acid strength. The value of pKa can be obtained by calculation using the following software package 1 based on a database of Hammett's substituent constants and publicly known literature values. All pKa values described in the present specification indicate values determined by calculation using this software package.

Software Package 1: Advanced Chemistry Development (ACD/Labs) Software V8.14 for Solaris (1994-2007 ACD/Labs)

A logP value can be obtained from the actual measurement using n-octanol and water; however, in the present invention, the partition coefficient (the ClogP value) calculated from the logP value estimation program is used. Specifically, the “ClogP value” in the present specification refers to a ClogP value obtained from “ChemBioDrow ultra ver.12”.

In describing a group (an atomic group) of the present specification, in a case where a description does not indicate substitution and non-substitution, the description means the group includes a group having a substituent as well as a group having no substituent. For example, the description “alkyl group” includes not only an alkyl group that does not have a substituent (an unsubstituted alkyl group) but also an alkyl group that has a substituent (a substituted alkyl group).

Further, the “organic group” in the present specification means a group containing at least one carbon atom.

Further, in the present specification, the kind of substituent, the position of substituent, and the number of substituents are not particularly limited in a case of being described as “may have a substituent”. The number of substituents may be, for example, one, two, three, or more. Examples of the substituent include a monovalent non-metal atomic group excluding a hydrogen atom, and for example, the following Substituent T can be selected.

(Substituent T)

Examples of Substituent T include halogen atoms such as a fluorine atom, a chlorine atom, a bromine atom, and an iodine atom; alkoxy group such as a methoxy group,an ethoxy group, and a tert-butoxy group; aryloxy groups such as a phenoxy group and a p-tolyloxy group; alkoxycarbonyl groups such as a methoxycarbonyl group, a butoxycarbonyl group, and a phenoxycarbonyl group; acyloxy groups such as an acetoxy group, a propionyloxy group, and a benzoyloxy group; acyl groups such as an acetyl group, a benzoyl group, an isobutyryl group, an acryloyl group, a methacryloyl group, and a methoxalyl group; alkylsulfanyl groups such as a methylsulfanyl group and tert-butylsulfanyl group; arylsulfanyl groups such as a phenylsulfanyl group and a p-tolylsulfanyl group; an alkyl group; a cycloalkyl group; an aryl group; a heteroaryl group; a hydroxy group; a carboxy group; a formyl group; a sulfo group; a cyano group; an alkylaminocarbonyl group; an arylaminocarbonyl group; a sulfonamide group; a silyl group; an amino group; a monoalkylamino group; a dialkylamino group; an arylamino group; and combinations thereof.

[Method of Producing Salt]

The method of producing a salt according to the embodiment of the present invention is a method of producing a salt, including,

reacting M⁺X⁻ with YH to generate XH and M⁺Y⁻, and subsequently removing the generated XH to obtain the M⁺Y³¹ ,

in which the M⁺X⁻is a salt of a cation represented by M⁺Y⁻, and an anion represented by X⁻,

the M⁺Y⁻ is a salt of the cation represented by M⁺ and an anion represented by Y⁻,

the XH is a conjugate acid of X⁻,

the YH is a conjugate acid of Y⁻,

the M⁺Y⁻ is a compound (a photo-acid generator) that generates an acid upon irradiation with an active ray or a radioactive ray,

a pKa of the XH is larger than a pKa of the YH, and

a ClogP value of the XH is larger than 2.

Examples of the preferred embodiment to which the above-described producing method according to the embodiment of the present invention is applied include the production of a salt containing a hydrophilic anion, with which metal impurities cannot be reduced by liquid separation purification (washing with water). In producing a salt (M⁺Y⁻) containing a hydrophilic anion (Y⁻), it is possible to reduce metal impurities by using a salt (M⁺X⁻) containing a more hydrophobic anion (X⁻), where the salt (M+X⁻) can be purified by liquid separation.

That is, in a case where a salt (M⁺X⁻) of which the metal impurity quantity has been reduced by liquid separation purification (washing with water) is used as a raw material and subjected to ion-exchange, it is also possible to reduce the metal impurity quantity of the obtained target product (M⁺Y⁻).

<pKa of XH and pKa of YH>

In the present invention, the pKa of XH is larger than the pKa of YH. That is, YH is a stronger acid than XH.

The pKa of XH is not particularly limited; however, it is preferably 6 to 12, more preferably 6.5 to 10.5, and still more preferably 7 to 8.

The pKa of YH is not particularly limited; however, it is preferably −11 to 8, more preferably −2 to 7, and still more preferably 0 to 6.

Further, the difference between the pKa of XH and YH, that is, (pKa of XH−pKa of YH) is preferably 0.5 or more, more preferably 1 or more, and still more preferably 2 or more.

<ClogP value of XH and ClogP value of YH>

In the present invention, the ClogP value of XH is larger than 2. That is, XH is hydrophobic, and X⁻ is also a hydrophobic anion. Since metal impurities are generally water-soluble, it is possible to reduce metal impurities by liquid separation purification (washing with water) from highly hydrophobic M⁺X⁻. Further, in the method of producing a salt according to the embodiment of the present invention, it is also possible to easily remove the produced XH by liquid separation in a case where the generated XH is removed to obtain M⁺Y⁻.

The ClogP value of YH is preferably smaller than the ClogP value of XH.

The ClogP value of YH is not particularly limited as long as it is smaller than the ClogP value of XH; however, for example, it is preferably 4 or less, more preferably 3 or less, still more preferably 2.5 or less, and particularly preferably 2 or less.

M⁺Y⁻ is a compound (a photo-acid generator) that generates an acid upon irradiation with an active ray or a radioactive ray, and, in particular, it is preferably a compound that generates an organic acid upon irradiation with an active ray or a radioactive ray. Further, M⁺X⁻ may be a photo-acid generator or not a photo-acid generator.

M⁺X⁻ and M⁺Y⁻ are preferably a sulfonium salt or an iodonium salt, and more preferably a sulfonium salt. That is, M⁺ is preferably a sulfonium ion or an iodonium ion, and more preferably a sulfonium ion.

<Structure of M⁺>

M⁺ is not particularly limited; however, it is preferably represented by, for example, General Formula (ZI) or General Formula (ZII).

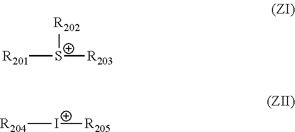

In General Formula (ZI), R₂₀₁, R₂₀₂, and R₂₀₃ each independently represent an organic group.

In General Formula (ZII), R₂₀₄ and R₂₀₅ each independently represent an organic group.

In General Formula (ZI), the number of carbon atoms of the organic group as R₂₀₁, R₂₀₂, and R₂₀₃ is generally 1 to 30 and preferably 1 to 20.

The organic group is not particularly limited; however, examples thereof include an alkyl group, an aryl group, a cycloalkyl group, and a heteroaryl group (a hetero atom thereof is preferably an oxygen atom, a nitrogen atom, a sulfur atom, or the like).

Further, two of R²⁰¹ to R²⁰³ may be bonded to form a ring structure, and the ring may contain an oxygen atom, a sulfur atom, an ester bond, an amide bond, or a carbonyl group. Examples of the group formed by bonding of two of R²⁰¹ to R²⁰³ include an alkylene group (for example, a butylene group or a pentylene group) and —CH₂—CH₂—O—CH₂—CH₂—.

At least one of R₂₀₁, R₂₀₂, or R203 is preferably an aryl group (preferably an aryl group having 6 to 10 carbon atoms, more preferably a phenyl group or a naphthyl group, and still more preferably a phenyl group).

In a case where at least one of R₂₀₁, R₂₀₂, or R₂₀₃ is an aryl group, all of R₂₀₁ to R₂₀₃ may be an aryl group, or part of R₂₀₁ to R₂₀₃ may be an aryl group and the rest thereof may be an alkyl group or a cycloalkyl group.

Further, one of R₂₀₁ to R₂₀₃ may be an aryl group, and the remaining two of R₂₀₁ to R₂₀₃ may be bonded to form a ring structure, where the ring may contain an oxygen atom, a sulfur atom, an ester group, an amide group, or a carbonyl group. Examples of the group formed by bonding of two of R₂₀₁ to R₂₀₃ include an alkylene group (for example, a butylene group, a pentylene group, or —CH₂—CH₂—O—CH₂—CH₂—) in which one or more methylene groups may be substituted with an oxygen atom, a sulfur atom, an ester group, an amide group, or a carbonyl group.

R₂₀₁ to R₂₀₃ may have a substituent. Examples of the substituent include the above-described Substituent T, and an alkoxy group or an alkyl group is preferable.

In General Formula (ZII), R₂₀₄ and R₂₀₅ each independently represent an organic group, and specific examples and preferable ranges thereof are the same as those of R₂₀₁, R₂₀₂, and R₂₀₃ in General Formula (ZI).

Preferred examples of M⁺ are shown below; however, they are not limited thereto.

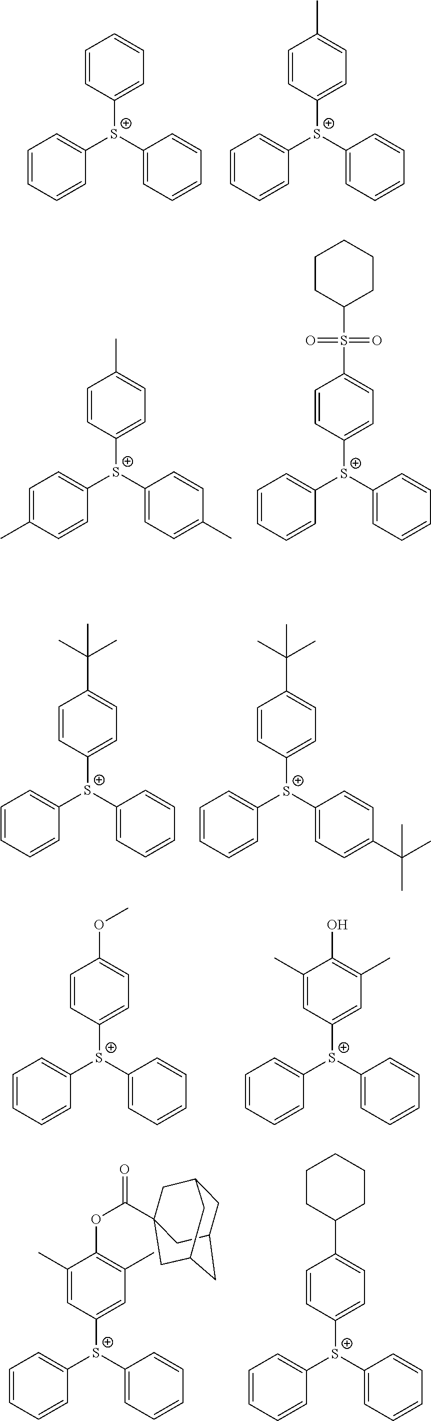

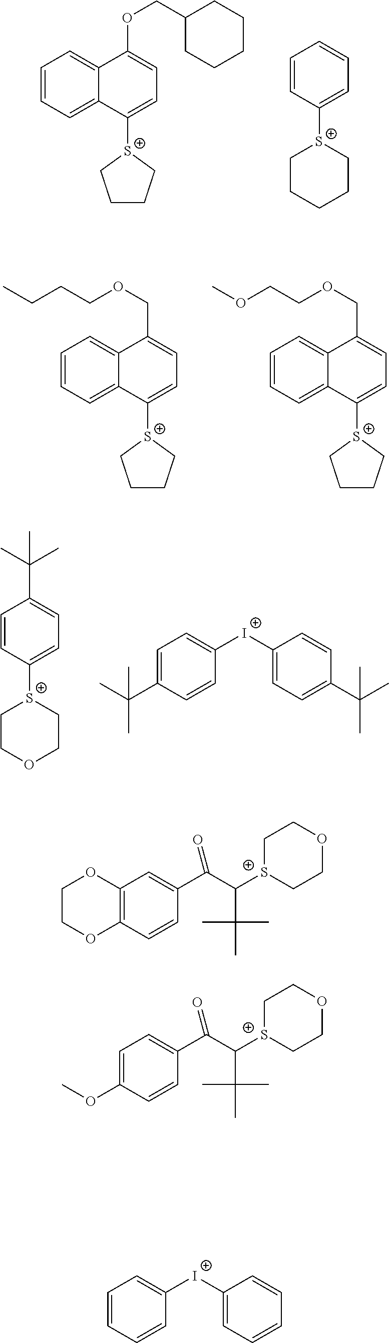

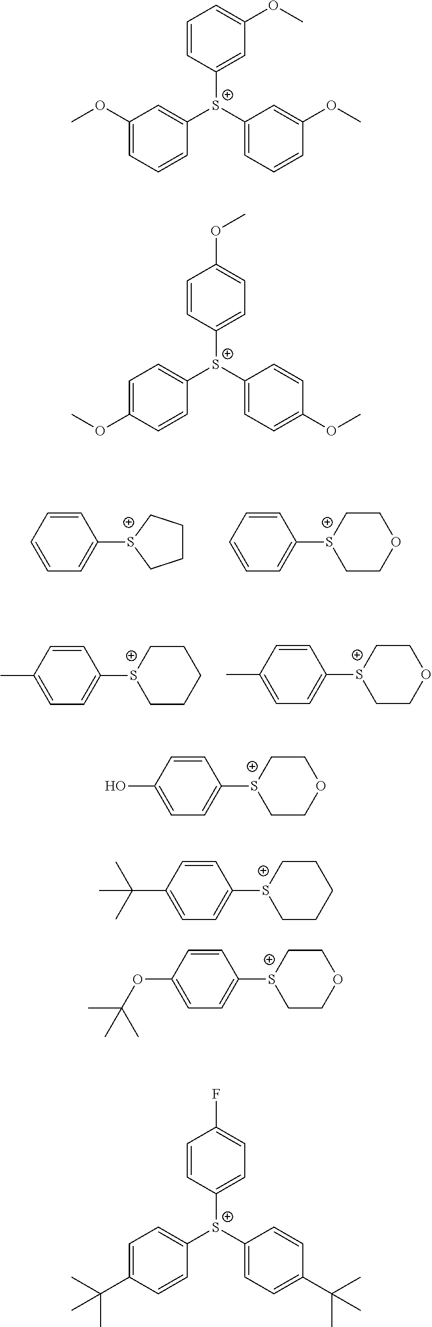

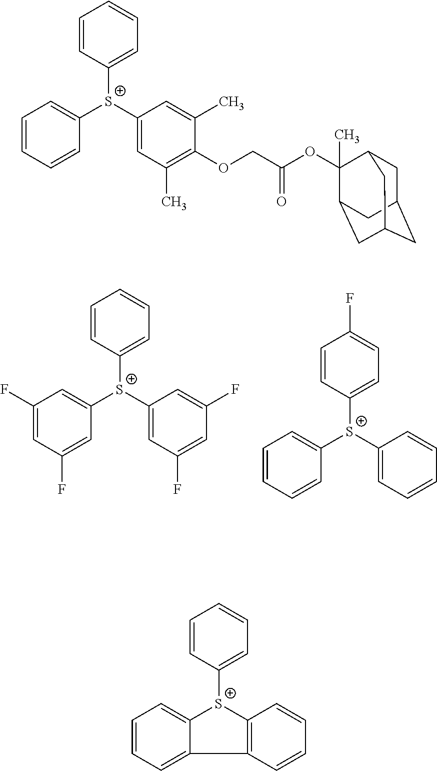 [SMILES: Cc1cc([S+](c2ccccc2)c2ccccc2)cc(C)c1OCC(=O)OC1(C)C2CC3CC(C2)CC1C3 ; Fc1cc(F)cc([S+](c2ccccc2)c2cc(F)cc(F)c2)c1 ; Fc1ccc([S+](c2ccccc2)c2ccccc2)cc1 ; c1ccc(-[s+]2c3ccccc3c3ccccc32)cc1]

<Structure of X⁻ and Y⁻>

In the present invention, X⁻ and Y⁻ are not particularly limited; however, they are each independently preferably an anion represented by any of General Formulae (1) to (6).

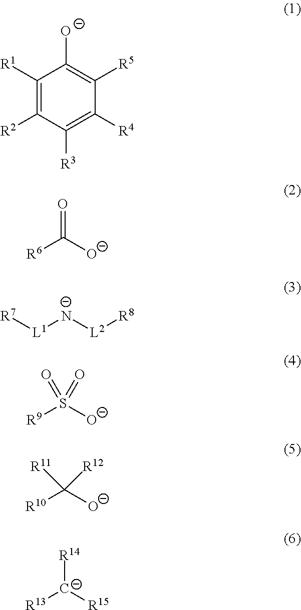

In General Formula (I), R¹ to R⁵ each independently represent a hydrogen atom, a halogen atom, a hydroxy group, or a monovalent organic group. Here, at least two of R¹, . . . , or R⁵ may be bonded to form a ring structure.

In General Formula (2), R⁶ represents a hydrogen atom, a halogen atom, a hydroxy group, or a monovalent organic group.

In General Formula (3), R⁷ and R⁸ each independently represent a hydrogen atom, a halogen atom, a hydroxy group, or a monovalent organic group. Here, R⁷ and R⁸ may be bonded to form a ring structure. L¹ represents —SO₂—, —C(═O)—, or a single bond, and L² represents —SO₂—, or —C(═O)—.

In General Formula (4), R⁹ represents a hydrogen atom, a halogen atom, a hydroxy group, or a monovalent organic group.

In General Formula (5), R¹⁰ to R¹² each independently represent a hydrogen atom, a halogen atom, a hydroxy group, or a monovalent organic group.

In General Formula (6), R¹³ to R¹⁵ each independently represent —SO₂—R¹⁶, —C(═O)—R¹⁶, or a cyano group. Here, R¹⁶ represents a hydrogen atom, a halogen atom, a hydroxy group, or a monovalent organic group.

Hereinafter, each General Formula will be described. In the present invention, X⁻ and Y⁻ are anions different from each other and each can be selected from R¹ to R¹⁶ in each General Formula in consideration of the above-described pKa of XH, pKa of YH, ClogP value of XH, and ClogP value of YH.

In General Formula (1), R¹ to R⁵ each independently represent a hydrogen atom, a halogen atom, a hydroxy group, or a monovalent organic group.

Examples of the halogen atom represented by R¹ to R⁵ include a fluorine atom, a chlorine atom, a bromine atom, and an iodine atom.

The monovalent organic group represented by R¹ to R⁵ is not particularly limited and may be a chain-like organic group or a cyclic organic group.

The chain-like organic group may be a chain-like hydrocarbon group or may be a hetero atom-containing group having an oxygen atom, a nitrogen atom, a sulfur atom, or the like between carbon atoms of a carbon-carbon bond. The chain-like hydrocarbon group and the hetero atom-containing group may be linear or branched.

The cyclic organic group may be a cyclic hydrocarbon group or a heterocyclic group having an oxygen atom, a nitrogen atom, a sulfur atom, or the like in the ring. The cyclic hydrocarbon group and the heterocyclic group may be an aliphatic group or an aromatic group.

The monovalent organic group represented by R¹ to R⁵ is preferably a chain-like hydrocarbon group or a cyclic hydrocarbon group.

Examples of the chain-like hydrocarbon group include an alkyl group, an alkenyl group, and an alkynyl group. The chain-like hydrocarbon group is preferably a chain-like hydrocarbon group having 1 to 10 carbon atoms, more preferably an alkyl group having 1 to 10 carbon atoms, and still more preferably an alkyl group having 1 to 5 carbon atoms.

Examples of the alkyl group having 1 to 5 carbon atoms include a methyl group, an ethyl group, an n-propyl group, an i-propyl group, an n-butyl group, a sec-butyl group, and a tert-butyl group.

The above alkyl group may have a substituent, and examples of the substituent include the above-described Substituent T. The substituent is preferably a hydroxy group or a halogen atom (preferably a fluorine atom). In a case where the alkyl group has a substituent and the substituent has a carbon atom, the carbon atom in the substituent is included in the above-described range of the number of carbon atoms. In R¹ to R¹⁶, the same treatment applies to other groups of which the preferred number of carbon atoms is described.

Examples of the cyclic hydrocarbon group include a cycloalkyl group, a cycloalkenyl group, a cycloalkynyl group, and an aryl group. The cyclic hydrocarbon group is preferably a cyclic hydrocarbon group having 3 to 20 carbon atoms and more preferably an aryl group having 6 to 20 carbon atoms.

Examples of the aryl group having 6 to 20 carbon atoms include a phenyl group, a naphthyl group, and an anthryl group, and a phenyl group or a naphthyl group is preferable, and a phenyl group is more preferable.

The aryl group may have a substituent, and examples of the substituent include the above-described Substituent T, and an alkyl group having 1 to 5 carbon atoms is preferable. The alkyl group as a substituent may further have a substituent, and examples thereof include a hydroxy group or a halogen atom (preferably a fluorine atom).

At least any two of R¹, . . . , or R⁵ may be bonded to form a ring structure. In this case, the ring structure to be formed is preferably a ring structure having 3 to 20 carbon atoms, more preferably a ring structure having 4 to 10 carbon atoms, and still more preferably a ring structure having 4 to 8 carbon atoms.

R¹ to R⁵ are preferably a hydrogen atom, a halogen atom, or an alkyl group having 1 to 10 carbon atoms, and are more preferably a hydrogen atom, a bromine atom, a fluorine atom, or an alkyl group having 1 to 5 carbon atoms, which is fluorine-substituted or unsubstituted.

In General Formula (2), R⁶ represents a hydrogen atom, a halogen atom, a hydroxy group, or a monovalent organic group.

The halogen atom represented by R⁶ is the same as the halogen atom as R¹ to R⁵ as described above.

The monovalent organic group represented by R⁶ is not particularly limited, and examples thereof include the same examples as those of the monovalent organic groups as R¹ to R⁵ described above.

The monovalent organic group represented by R⁶ is preferably a chain-like hydrocarbon group or a cyclic hydrocarbon group.

Examples of the chain-like hydrocarbon group represented by R⁶ include the same examples as those of the chain-like hydrocarbon groups as R¹ to R⁵ described above, and the same applies to the preferred examples thereof.

Examples of the cyclic hydrocarbon group represented by R⁶ include the same examples as those of the cyclic hydrocarbon groups as R¹ to R⁵ described above, and the same applies to the preferred examples thereof.

R⁶ is preferably a hydrogen atom, a hydroxy group, an alkyl group having 1 to 10 carbon atoms, or an aryl group having 6 to 20 carbon atoms, and is more preferably a hydrogen atom, a hydroxy group, an alkyl group having 1 to 5 carbon atoms, which is substituted or unsubstituted, or a phenyl group which is substituted with a fluorinated alkyl group.

In General Formula (3), R⁷ and R⁸ each independently represent a hydrogen atom, a halogen atom, a hydroxy group, or a monovalent organic group.

Examples of the halogen atom represented by R⁷ and R⁸ include the same examples as those of the halogen atom as R¹ to R⁵ as described above.

The monovalent organic group represented by R⁷ and R⁸ is not particularly limited, and examples thereof include the same groups as the monovalent organic groups as R¹ to R⁵ described above.

The monovalent organic group represented by R⁷ and R⁸ is preferably a chain-like hydrocarbon group or a cyclic hydrocarbon group.

Examples of the chain-like hydrocarbon group represented by R⁷ and R⁸ include the same examples as those of the chain-like hydrocarbon group as R¹ to R⁵ described above, and the same applies to the preferred examples thereof.

Examples of the cyclic hydrocarbon group represented by R⁷ and R⁸ include the same examples as those of the chain-like hydrocarbon group as R¹ to R⁵ described above, and the same applies to the preferred examples thereof.

Here, R⁷ and R⁸ may be bonded to form a ring structure. In this case, the ring structure to be formed is a ring structure so that the anion moiety of the compound represented by General Formula (3) is more preferably a ring structure having 4 to 10 carbon atoms and still more preferably a ring structure having 4 to 8 carbon atoms.

The group formed by the bonding of R⁷ and R⁸ is preferably an alkylene group which is fluorine-substituted or unsubstituted.

R⁷ and R⁸ are preferably an alkyl group having 1 to 10 carbon atoms or preferably bonded to form a ring structure, and are more preferably an alkyl group having 1 to 5 carbon atoms, which is fluorine-substituted or unsubstituted, or more preferably bonded so that the anion moiety of the compound represented by General Formula (3) is a ring structure having 4 to 8 carbon atoms.

It is preferable that at least one of L¹ or L² represents —SO₂—, and it is more preferable that both L¹ and L² represent —SO₂—.

In General Formula (4), R⁹ represents a hydrogen atom, a halogen atom, a hydroxy group, or a monovalent organic group.

Examples of the halogen atom represented by R⁹ include the same examples as those of the halogen atom as R¹ to R⁵ as described above.

The monovalent organic group represented by R⁹ is not particularly limited, and examples thereof include the same group as the monovalent organic group as R¹ to R⁵ described above.

The monovalent organic group represented by R⁹ is preferably a chain-like hydrocarbon group or a cyclic hydrocarbon group.

Examples of the chain-like hydrocarbon group represented by R⁹ include the same examples as those of the chain-like hydrocarbon group as R¹ to R⁵ described above, and the same applies to the preferred examples thereof.

Examples of the cyclic hydrocarbon group represented by R⁹ include the same examples as those of the cyclic hydrocarbon groups as R¹ to R⁵ described above, and the same applies to the preferred examples thereof.

R⁹ is preferably an alkyl group having 1 to 10 carbon atoms, or an aryl group having 6 to 20 carbon atoms and is more preferably an alkyl group having 1 to 5 carbon atoms, which is fluorine-substituted or unsubstituted, or a phenyl group which is substituted with an alkyl group or unsubstituted.

In General Formula (5), R¹⁰ to R¹² each independently represent a hydrogen atom, a halogen atom, a hydroxy group, or a monovalent organic group.

Examples of the halogen atom represented by R¹⁰ to R¹² include the same examples as those of the halogen atom as R¹ to R⁵ as described above.

The monovalent organic group represented by R¹⁰ to R¹² is not particularly limited, and examples thereof include the same group as the monovalent organic group as R¹ to R⁵ described above.

The monovalent organic group represented by R¹⁰ to R¹² is preferably a chain-like hydrocarbon group or a cyclic hydrocarbon group.

Examples of the chain-like hydrocarbon group represented by R¹⁰ to R¹² include the same examples as those of the chain-like hydrocarbon group as R¹ to R⁵ described above, and the same applies to the preferred examples thereof.

Examples of the cyclic hydrocarbon group represented by R¹⁰ to R¹² include the same examples as those of the cyclic hydrocarbon group as R¹ to R⁵ described above, and the same applies to the preferred examples thereof.

R¹⁰ to R¹² each independently more preferably represent a monovalent organic group, still more preferably an alkyl group having 1 to 10 carbon atoms or an aryl group having 6 to 20 carbon atoms, and particularly preferably an alkyl group having 1 to 5 carbon atoms, which is fluorine-substituted.

In General Formula (6), R¹³ to R¹⁵ each independently represent —SO₂—R¹⁶, —C(═O)—R¹⁶, or a cyano group.

R¹³ to R¹⁵ are each independently preferably —SO₂—R¹⁶ or a cyano group, and is more preferably —SO₂—R¹⁶.

Here, R¹⁶ represents a hydrogen atom, a halogen atom, a hydroxy group, or a monovalent organic group.

Examples of the halogen atom represented by R¹⁶ include the same examples as those of the halogen atom as R¹ to R⁵ as described above.

The monovalent organic group represented by R¹⁶ is not particularly limited, and examples thereof include the same group as the monovalent organic group as R¹ to R⁵ described above.

The monovalent organic group represented by R¹⁶ is preferably a chain-like hydrocarbon group or a cyclic hydrocarbon group.

Examples of the chain-like hydrocarbon group represented by R¹⁶ include the same examples as those of the chain-like hydrocarbon group as R¹ to R⁵ described above, and the same applies to the preferred examples thereof.

Examples of the cyclic hydrocarbon group represented by R¹⁶ include the same examples as those of the cyclic hydrocarbon groups as R¹ to R⁵ described above, and the same applies to the preferred examples thereof.

R¹⁶ more preferably represents a monovalent organic group, still more preferably an alkyl group having 1 to 10 carbon atoms or an aryl group having 6 to 20 carbon atoms, and particularly preferably an alkyl group having 1 to 5 carbon atoms, which is fluorine-substituted.

X⁻ is preferably an anion represented by General Formula (1) or (3), and is more preferably an anion represented by General Formula (1).

It is preferable that X⁻ is represented by General Formula (1) and at least one of R¹, . . . , or R⁵ in General Formula (1) represents a monovalent organic group.

Y⁻ is preferably,

an anion represented by General Formula (1), and R¹ to R⁵ in General Formula (1) represent a halogen atom,

an anion represented by General Formula (2), and R⁶ in General Formula (2) represents a hydroxy group or a monovalent organic group,

an anion represented by General Formula (3), and R⁷ and R⁸ in General Formula (3) represent a monovalent organic group,

an anion represented by General Formula (4), and R⁹ in General Formula (4) represents a monovalent organic group,

an anion represented by General Formula (5), and R¹⁰ to R¹² in General Formula (5) each independently represent a monovalent organic group, or

an anion represented by General Formula (6), and R¹³ or R¹⁵ in General Formula (6) each independently represent —SO₂—R¹⁶, where R¹⁶ represents a monovalent organic group.

Specific examples of M⁺X⁻ or M⁺Y⁻, where X⁻ or Y⁻ is represented by any one of General Formulae (1) to (6) are shown below; however, the present invention is not limited thereto. In the specific examples below, Me represents a methyl group.

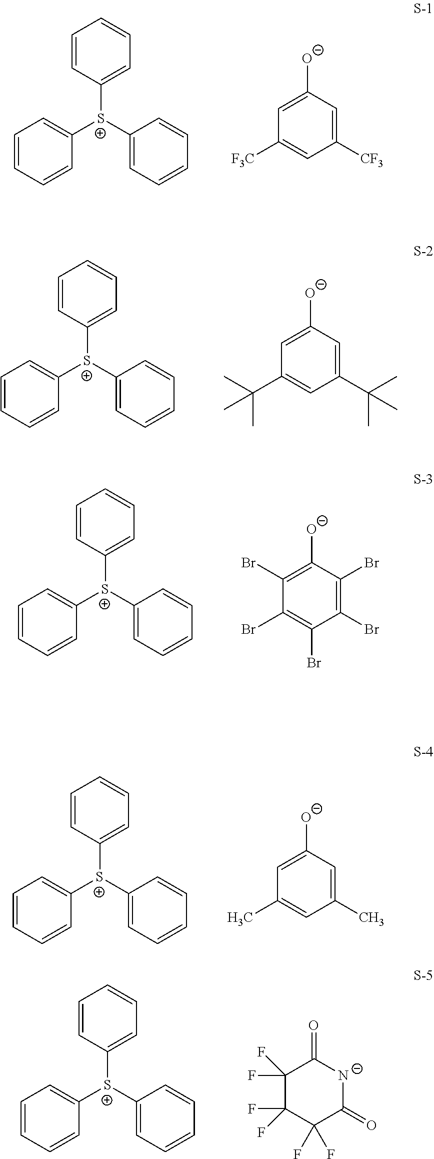

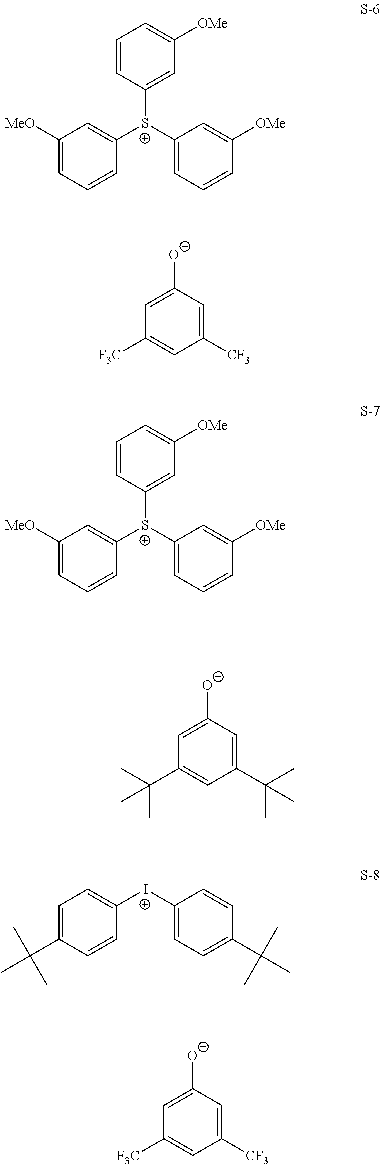

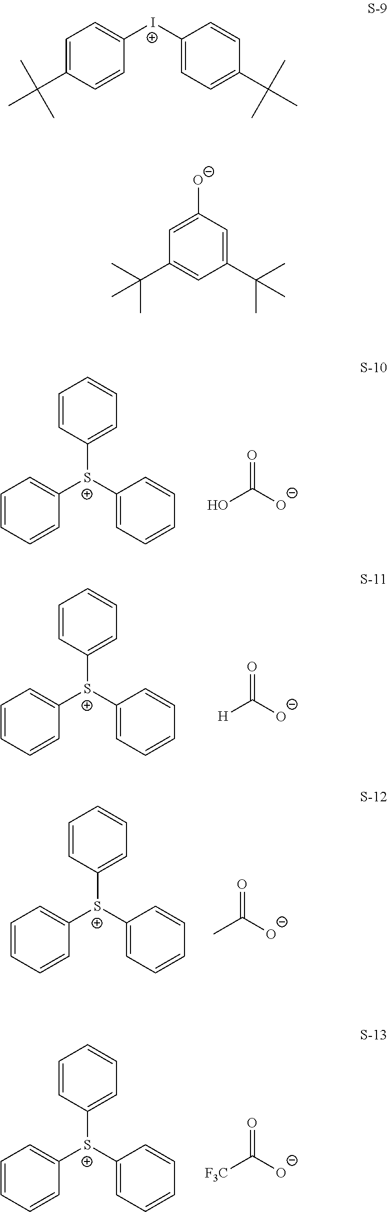

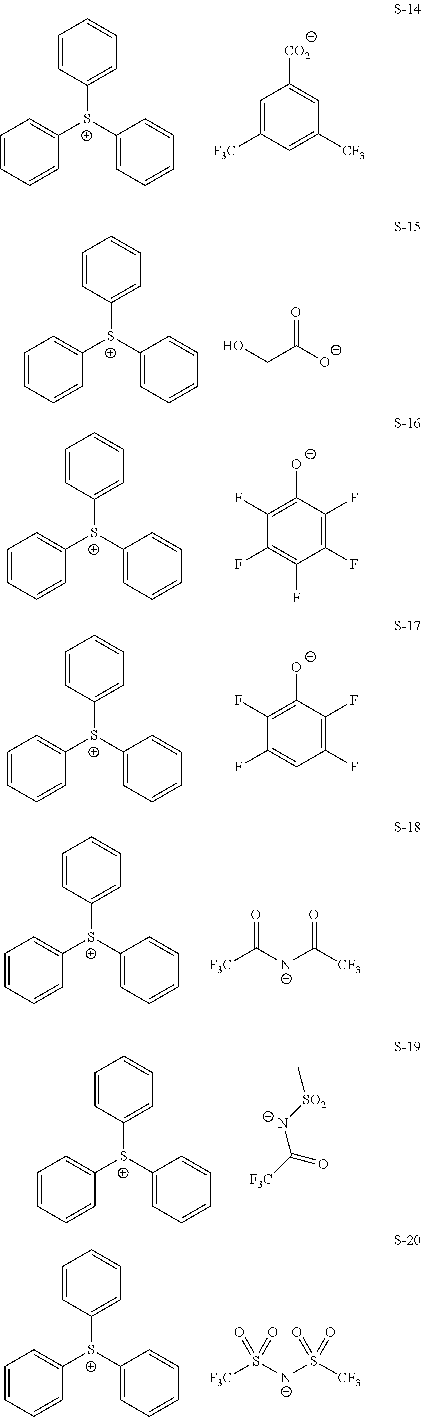

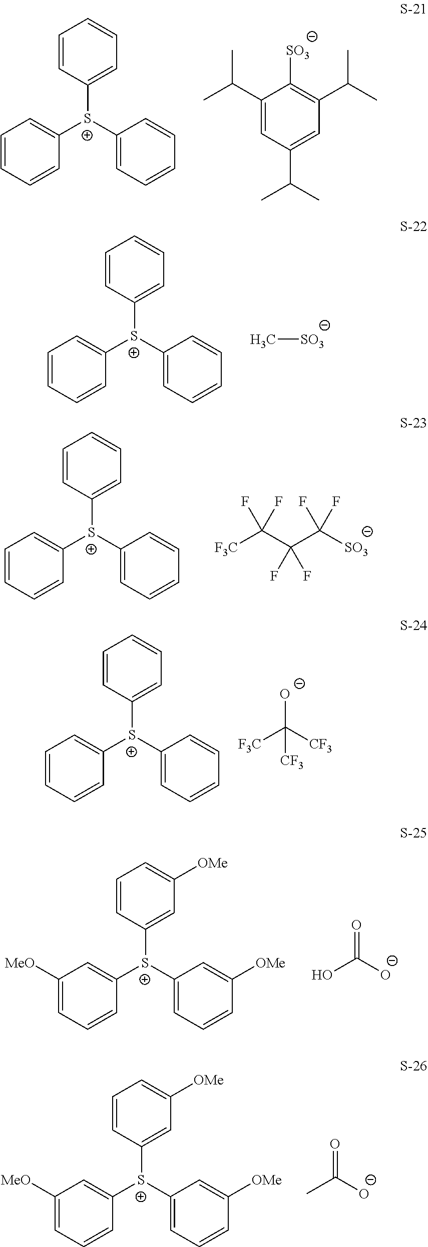

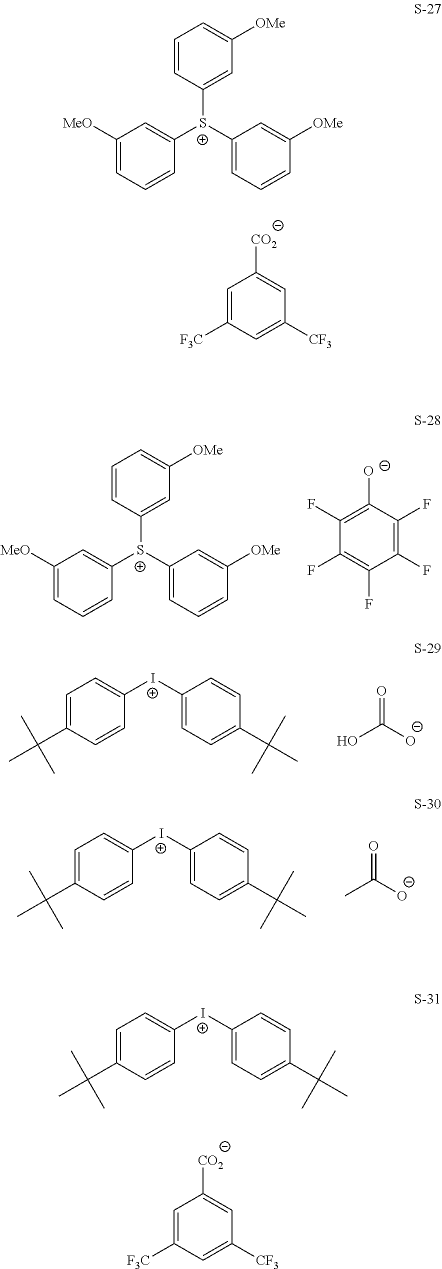

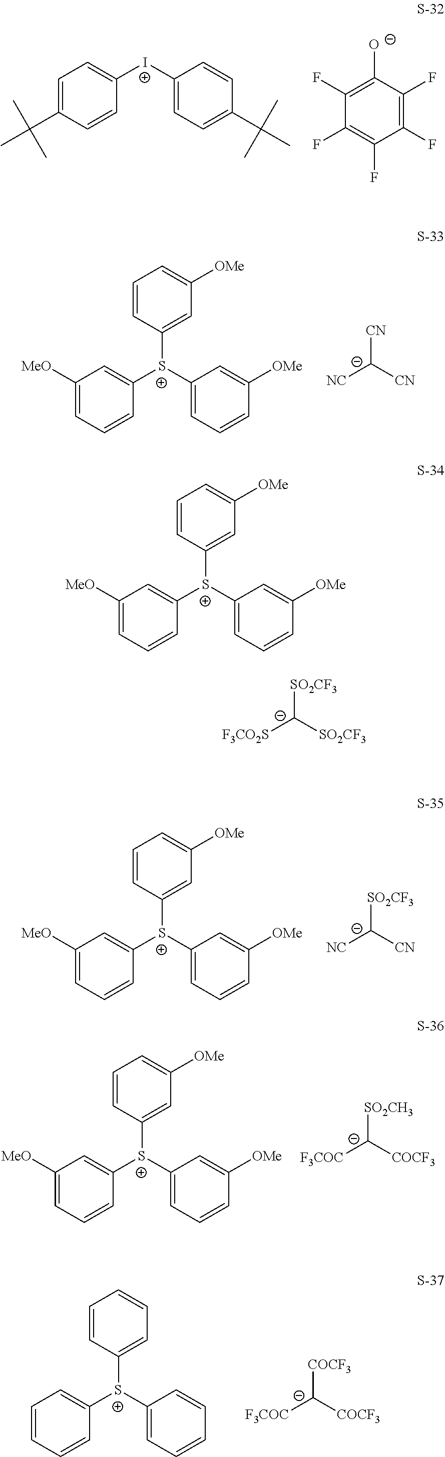

Preferred examples of M⁺X⁻ include compounds S-1 to S-9.

Preferred examples of M⁺Y⁻ include compounds S-10 to S-32.

<Reaction of M⁺X⁻ with YH>

The reaction of M⁺X⁻ with YH in the method of producing a salt according to the embodiment of the present invention will be described.

The amount of YH to used with respect to M⁺X⁻ is not particularly limited, and, for example, it is usually 0.8 to 10 molar equivalents, preferably 0.8 to 5 molar equivalents, and more preferably 0.9 to 2 molar equivalents with respect to the substance amount (mol) of M⁺X⁻.

The reaction of M⁺X⁻ with YH is preferably carried out in a reaction solvent.

Examples of the preferred reaction solvent include an ether-based solvent, an ester-based solvent, a ketone-based solvent, a nitrile-based solvent, an alcohol-based solvent, or a fluorine-based solvent.

The ether-based solvent is preferably an ether-based solvent having 1 to 10 carbon atoms, and more preferably methyl tert-butyl ether (MTBE), diisopropyl ether, cyclopentyl methyl ether (CPME), or tetrahydrofuran (THF).

The ester-based solvent is preferably an ester-based solvent having 1 to 10 carbon atoms and more preferably ethyl acetate.

The ketone-based solvent is preferably a ketone-based solvent having 1 to 10 carbon atoms and more preferably acetone.

The nitrile-based solvent is preferably a nitrile-based solvent having 1 to 5 carbon atoms and more preferably acetonitrile.

The alcohol-based solvent is preferably an alcohol-based solvent having 1 to 5 carbon atoms and more preferably methanol, ethanol, or isopropanol.

The fluorine-based solvent is preferably a fluorine-based solvent having 1 to 5 carbon atoms and more preferably hexafluoroisopropanol (HFIP).

The reaction solvent is preferably an ether-based solvent, a ketone-based solvent, or a nitrile-based solvent, more preferably an ether-based solvent, and still more preferably CPME or THF.

As the reaction solvent, one kind of solvent may be used alone, or two or more kinds of solvents may be used in combination.

The amount of the reaction solvent to be used is not particularly limited, and it is usually 0.1 to 20 mL, preferably 0.5 to 10 mL, and more preferably 1 to 5 mL with respect to 1 mmol M⁺X⁻.

The reaction temperature of the above reaction in the producing method according to the embodiment of the present invention is preferably −78° C. to 100° C. and more preferably 0° C. to 40° C. from the viewpoint of the reaction efficiency and the yield of the target product M⁺Y⁻.

The pressure at the time of reaction of the above reaction in the producing method according to the embodiment of the present invention is not particularly limited as long as a series of reactions is carried out without delay, and, for example, the above reaction may be carried out at normal pressure.

The reaction time of the above reaction in the producing method according to the embodiment of the present invention is not particularly limited, and the preferred reaction time varies depending on the kinds and the amounts of the components to be used, the kind of reaction solvent, the reaction temperature, the pressure at the time of reaction, and the like; however, for example, it is generally 1 minute to 10 hours and preferably 1 minute to 5 hours.

In the producing method according to the embodiment of the present invention, XH and M⁺Y⁻ are generated by the reaction of M⁺X⁻ with YH. Then, XH is removed to obtain the target product M⁺Y⁻.

In the present invention, M⁺Y⁻ can be isolated by removing by-products such as XH and a reaction solvent by a general post-treatment operation or purification operation.

As a specific example of the isolation method, the isolation can be carried out by a filtration operation in a case where M⁺Y⁻ is obtained as a crystal. In a case where M⁺Y⁻ is not obtained as a crystal, the target product M⁺Y⁻ can be isolated as an oily product by concentration under reduced pressure.

In the present invention, it is preferable that a crystal containing M or an oily product containing M⁺Y⁻ is obtained by the reaction of M⁺X⁻ with YH, the crystal is washed with a washing solvent or the oily product is concentrated under reduced pressure, and XH contained in the crystal or the oily product is removed. In this manner, XH in the target product can be further reduced.

The solvent that is used for washing is preferably an ether-based solvent, an ester-based solvent, a ketone-based solvent, a nitrile-based solvent, an alcohol-based solvent, or a fluorine-based solvent. Specific examples and preferable examples of each solvent include the same examples as those of the above-described reaction solvent.

The content of metal impurities in the target product M⁺Y⁻ is preferably 10 parts per million (ppm) or less and more preferably less than 2 ppm based on the mass. In a case where the content thereof is less than 2 ppm, the target product M⁺Y⁻ can be used even in fields where metal content standards are strict, such as in the field of semiconductor manufacturing, which is preferable.

The content of metal impurities can be measured by an inductively coupled plasma (ICP) emission spectrophotometer.

In particular, in the present invention, it is preferable that each of the contents of sodium, calcium, and silver in the target product M⁺Y⁻ is in the above range.

<Preparation of M⁺X⁻>

The method for preparing M⁺X⁻, which is one of the raw materials for the reaction in the method of producing a salt according to the embodiment of the present invention, is not particularly limited; however, for example, the following method is preferable.

That is, in the present invention, it is preferable that M⁺G⁻ is reacted with Q⁺X⁻ to generate M⁺X⁻ and Q⁺G⁻, and then, the generated Q⁺G⁻ is removed to obtain M⁺X⁻.

Here, G⁻ is a halogen ion, and Q⁺ is an alkali metal ion.

M⁺G⁻ is typically a halogenated onium salt, and the cation (M³⁰ ) is the same as that described above.

Examples of G⁻ include a fluorine ion (F⁻), a chlorine ion (Cl⁻), a bromine ion (Br⁻), an iodine ion (I⁻), and from the viewpoint of solubility in water, a chlorine ion or a bromine ion is preferable.

M⁺G⁻ is preferably triphenylsulfonium chloride, triphenylsulfonium bromide, or trimethoxytriphenylsulfonium bromide, and more preferably triphenylsulfonium bromide.

The anion (X⁻) in Q⁺X⁻ is the same as that described above.

Examples of Q⁺ include a lithium ion (Li⁺), a sodium ion (Na⁺), and a potassium ion (K⁺).

Q⁺X⁻ can be obtained by a known method.

In the reaction of M⁺G⁻ with Q⁺X⁻, the amount of Q⁺X⁻ to be used with respect to M⁺G⁻ is not particularly limited as long as it is a practical amount, and for example, it is usually 0.5 to 2 molar equivalents, preferably 0.7 to 1.5 molar equivalents, and more preferably 0,7 to 1.2 molar equivalents with respect to the substance amount (mol) of M⁺G⁻.

The reaction time of the reaction of M⁺G⁻ with Q⁺X⁻ is not particularly limited, and the preferred reaction time varies depending on the kinds and the amounts of the components to be used, the kind of solvent, the reaction temperature, the pressure at the time of reaction, and the like; however, for example, it is generally 1 minute to 5 hours and more preferably 1 minute to 2 hours.

The reaction temperature and the reaction pressure are not particularly limited, and the reaction can be carried out at, for example, normal temperature and normal pressure.

In the present invention, it is preferable that the reaction of M⁺G⁻ with Q⁺X⁻ is carried out in the presence of an organic solvent and water, and the obtained organic layer is washed with water to obtain M⁺X⁻.

In the related art, it was common to synthesize M⁺Y⁻, which is the target product of the present invention, directly from M⁺G⁻ by ion exchange; however, in a case where both M⁺G⁻ and M⁺Y⁻ were hydrophilic salts, the metal content could not be reduced by washing with water.

On the other hand, in the preferred embodiment of the present invention, M⁺X⁻ is synthesized from M⁺G⁻, and then M⁺Y⁻ is synthesized from M⁺X⁻ as described above. That is, M⁺X⁻ is synthesized as an intermediate, and using this, M⁺X⁻ is synthesized. M⁺G⁻ is hydrophilic, whereas M⁺X⁻ is hydrophobic. Accordingly, the reaction of M⁺G⁻ with Q⁺X⁻ can be carried out in the presence of an organic solvent and water, and the obtained organic layer (where most of M⁺X⁻ is present) can be purified by liquid separation (washed with water) to obtain M⁺X⁻ in which the metal content is reduced.

The organic solvent in the reaction of M⁺G⁻ with Q⁺X⁻ is not particularly limited; however, for example, dichloromethane, chloroform, or ethyl acetate is preferable, and dichloromethane is more preferable. The organic solvent may be used alone, or two or more thereof may be used in combination.

The amounts of the organic solvent to be used and the water to be used are not particularly limited, and they are usually 0.1 to 20 mL, preferably 0.5 to 10 mL, and more preferably 1 to 5 mL with respect to 1 mmol M⁺G⁻.

The mixing ratio between the organic solvent and water is not particularly limited.

It is also possible to remove Q⁺G⁻ and purify M⁺X⁻ by a conventional method.

### EXAMPLES

Hereinafter, the present invention will be described in more detail based on Examples. The materials, amounts of use, proportions, treatments, procedures, and the like described in the following Examples can be appropriately modified as long as the gist of the invention is maintained. Therefore, the scope of the present invention should not be restrictively interpreted by the following Examples.

### Example 1

20 g (87 mmol) of 3,5-bis(trifluoromethyl)phenol (BisCF₃PhOH), 6.97 g (87 mmol) of NaOH (a 50% by mass aqueous solution), and water (50 mL) were mixed and stirred at 25° C. for 30 minutes. Then, 38.8 g (113 mmol) of triphenylsulfonium bromide (TPSBr), dichloromethane (200 mL), and water (50 mL) were added thereto and mixed with a 1,000 mL separatory funnel. The organic layer was washed once with 0.01 mol/L HCl (200 mL) and four times with water (200 mL), and the washed organic layer was concentrated to obtain 44.7 g of an intermediate (a compound A-1).

Subsequently, 5.0 g (10.2 mmol) of the compound A-1 was dissolved in cyclopentyl methyl ether (CPME) (50 mL), and 270 mg of water was further added thereto. Carbon dioxide was blown into the reaction system, and the mixture was stirred at 25° C. for 1 hour and 30 minutes. The generated crystals were filtered and washed twice with CPME (30 mL) to obtain 1.49 g (yield: 45%) of white crystals. As a result of analysis by ¹H-NMR and ¹³C-NMR, it was confirmed that the white crystals were a hydrogen carbonate triphenylsulfonium salt (a compound B-1). The results of ¹ H-NMR and ¹³C-NMR are shown below.

¹H-NMR (400 MHz, D₂O): 7.77 to 7.60 (15H, m)

¹³C-NMR (400 MHz, D₂O): 124.1, 130.7, 131.4, 134.7, 160.3

The reaction scheme of Example 1 is shown below. In Example 1, first, TPSBr (M⁺G⁻) is reacted with a sodium salt of 3,5-bis(trifluoromethyl)phenol (Q⁺X⁻) to generate the compound A-1 (M⁺X⁻) and NaBr (Q⁺G⁻). Here, the compound A-1 (M⁺X⁻) is mainly present in the organic layer, and NaBr (Q⁺G⁻) is mainly present in the aqueous layer. Accordingly, NaBr (Q⁺G⁻) can be removed by liquid separation to obtain the compound A-1 (M⁺X⁻). Furthermore, in a case where the organic layer is washed with water, NaBr (Q⁺G⁻) slightly present in the organic layer can be removed, and thus the purity of the compound A-1 (M⁺X⁻) in the organic layer can be increased. Thereafter, the compound A-1 (M⁺X⁻) is reacted with carbonic acid (YH) to generate the compound B-1 (M⁺Y⁻) and 3,5-bis(trifluoromethyl)phenol. Here, the compound B-1 (M⁺Y⁻) becomes a crystal and becomes separated from 3,5-bis(trifluoromethyl)phenol (XH), and thus it is possible to remove 3,5-bis(trifluoromethyl)phenol (XH) to obtain the target product, the compound B-1 (M⁺Y⁻). Furthermore, in a case where the crystals are washed with CPME, a slight amount of 3,5-bis(trifluoromethyl)phenol (XH) present in the crystals can be removed, and thus the compound B-1 (M⁺Y⁻) having more high purity can be obtained.

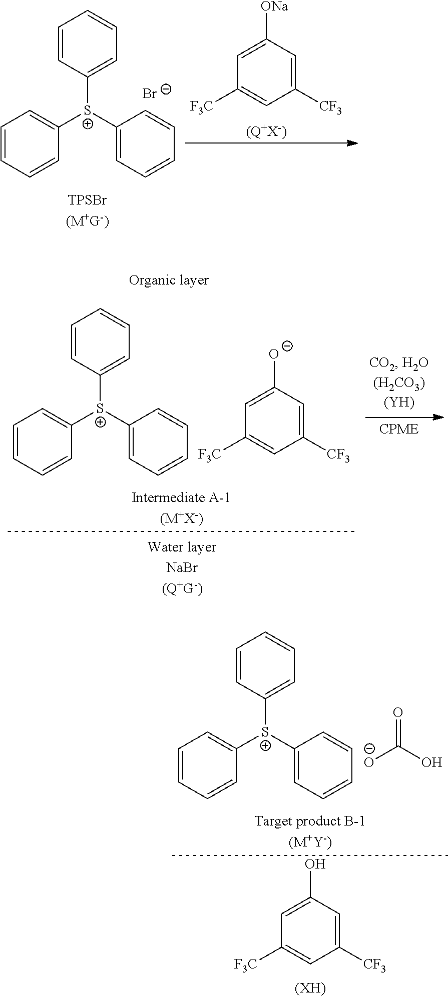

### Example 2

The intermediate (the compound A-1) was synthesized in the same manner as in Example 1, then 5.0 g (10.2 mmol) of the compound A-1 was dissolved in tetrahydrofuran (THF) (50 mL), and further, 5.0 g (10.2 mmol) of acetic acid was added thereto, and the mixture was stirred at 25° C. for 2 hours. The reaction solution was concentrated under reduced pressure with a rotary evaporator and heated to 80° C. under reduced pressure conditions to remove 3,5-bis(trifluoromethyl)phenol, and 2.80 g (yield: 90%) of an oily product was obtained. As a result of analysis by ¹H-NMR, it was confirmed that the obtained oily product was an acetate triphenylsulfonium salt (the compound B-2). The result of ¹H-NMR is shown below.

¹H-NMR (400 MHz, DMSO-d₆): 7.89 to 7.77 (15H, m), 1.54 (3H, ₛ)

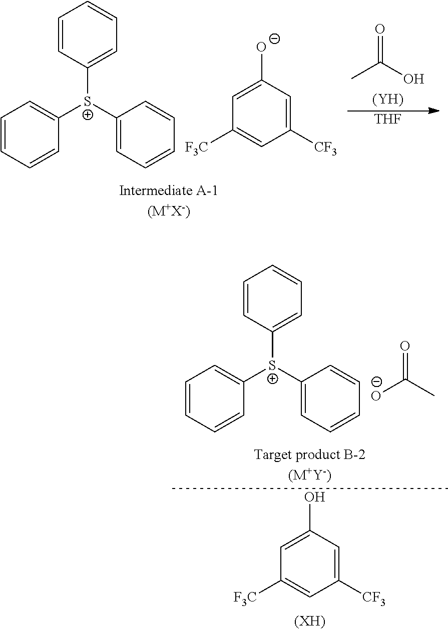

### Example 3

The intermediate (the compound A-1) was synthesized in the same manner as in Example 1, then 5.0 g (10.2 mmol) of the compound A-1 was dissolved in tetrahydrofuran (THF) (50 mL), and further, 1.2 g (10.2 mmol) of trifluoroacetic acid was added thereto, and the mixture was stirred at 25° C. for 2 hours. The reaction solution was concentrated under reduced pressure with a rotary evaporator and heated to 80° C. under reduced pressure conditions to remove 3,5-bis(trifluoromethyl)phenol, and 2.9 g (yield: 75%) of an oily product was obtained. As a result of analysis by ¹H-NMR and ¹⁹F-NMR, it was confirmed that the obtained oily product was a trifluoroacetate triphenylsulfonium salt (the compound B-3).

### Example 4

The intermediate (the compound A-1) was synthesized in the same manner as in Example 1, then 5.0 g (10.2 mmol) of the compound A-1 was dissolved in tetrahydrofuran (THF) (50 mL), and further, 2.6 g (10.2 mmol) of 3,5-bis(trifluoromethyl)benzoic acid was added thereto, and the mixture was stirred at 25° C. for 3 hours. The reaction solution was concentrated under reduced pressure with a rotary evaporator and heated to 80° C. under reduced pressure conditions to remove 3,5-bis(trifluoromethyl)phenol, and 4.5 g (yield: 85%) of an oily product was obtained. As a result of the analysis by ¹H-NMR and ¹⁹F-NMR, it was confirmed that the obtained oily product was a 3,5-bis(trifluoromethyl)benzoate triphenylsulfonium salt (a compound B-4).

### Example 5

The intermediate (the compound A-1) was synthesized in the same manner as in Example 1, then 5.0 g (10.2 mmol) of the compound A-1 was dissolved in THF (50 mL), and further, 1.88 g (10.2 mmol) of pentafluorophenol was added thereto, and the mixture was stirred at 25° C. for 3 hours. The reaction solution was concentrated under reduced pressure with a rotary evaporator and heated to 80° C. under reduced pressure conditions to remove 3,5-bis(trifluoromethyl)phenol, and 3.5 g (yield: 78%) of an oily product was obtained. As a result of the analysis by ¹H-NMR and ¹⁹F-NMR, it was confirmed that the obtained oily product was a pentafluorophenol triphenylsulfonium salt (the compound B-5). The results of ¹H-NMR and ¹⁹F-NMR are shown below.

¹H-NMR (400 MHz, DMSO-d₆): 7.89 to 7.76 (15H, m)

¹⁹F-NMR (400 MHz, DMSO-d₆): −171.9 (2F, m), −172.2 (2F, m), −196.2 (1F, m)

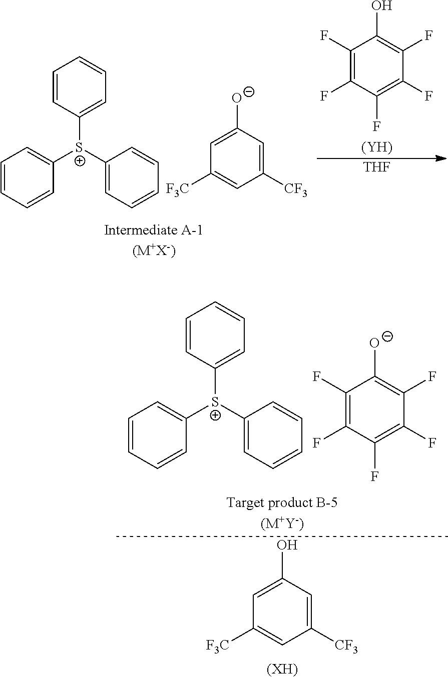

### Example 6

The intermediate (the compound A-1) was synthesized by the same method as in Example 1, and subsequently, in the same manner as in Example 2, the compound A-1 (10.2 mmol) was reacted with bistrifluoroacetamide (10.2 mmol), and the purification was followed, thereby obtaining an oily product at a yield of 80%. As a result of the analysis by ¹H-NMR and ¹⁹F-NMR, it was confirmed that the obtained oily product was a compound B-6. The results of ¹H-NMR and ¹⁹F-NMR are shown below.

¹H-NMR (400 MHz, DMSO-d₆): 7.89 to 7.76 (15H, m)

¹⁹F-NMR (400 MHz, DMSO-d₆): −76.0 (6F, s)

### Example 7

The intermediate (the compound A-1) was synthesized by the same method as in Example 1, and subsequently, in the same manner as in Example 2, the compound A-1 (10.2 mmol) was reacted with N-(trifluorornethanesulfonyl)trifluoroacetamide (10.2 mmol), and the purification was followed, thereby obtaining an oily product at a yield of 75%. As a result of the analysis by ¹H-NMR, it was confirmed that the obtained oily product was a compound B-7. The result of ¹H-NMR is shown below.

¹H-NMR (400 MHz, DMSO-d₆): 2.84 (3H, s), (7.89 to 7.76 (15H, m))

### Example 8

The intermediate (the compound A-1) was synthesized by the same method as in Example 1, and subsequently, in the same manner as in Example 2, the compound A-1 (10.2 mmol) was reacted with bistrifluoromethanesulfonylimide (10.2 mmol), and the purification was followed, thereby obtaining an oily product at a yield of 60%. As a result of the analysis by ¹H-NMR and ¹⁹F-NMR, it was confirmed that the obtained oily product was a compound B-8. The results of ¹H-NMR and ¹⁹F-NMR are shown below.

¹H-NMR (400 MHz, DMSO-d₆): 7.89 to 7.76 (15H, m)

¹⁹F-NMR (400 MHz, DMSO-d₆): −80.4 (6F, s)

### Example 9

The intermediate (the compound A-1) was synthesized by the same method as in Example 1, and in the same manner as in Example 1, the compound A-1 (10.2 mmol) was subsequently reacted with 2,4,6-triisopropyl beneznesulfonic acid (10.2 mmol), and the purification was followed, thereby obtaining an oily product at a yield of 82%. As a result of the analysis by ¹H-NMR, it was confirmed that the obtained oily product was a compound B-9. The result of ¹H-NMR is shown below.

¹H-NMR (400 MHz, DMSO-d₆): 1.08 (12H, d), 1.15 (6H, d), 2.78 (1H, m), 4.57 (2H, m), 6.93 (2H, s), 7.89 to 7.76 (15H, m)

### Example 10

The intermediate (the compound A-1) was synthesized by the same method as in Example 1, and in the same manner as in Example 1, the compound A-1 (10.2 mmol) was subsequently reacted with nonafluoro-tert-butanol (10.2 mmol), and the purification was followed, thereby obtaining an oily product at a yield of 55%. As a result of the analysis by ¹H-NMR and ¹⁹F-NMR, it was confirmed that the obtained oily product was a compound B-10. The results of ¹H-NMR and ¹⁹F-NMR are shown below.

¹H-NMR (400 MHz, DMSO-d₆): 7.89 to 7.76 (15H, m)

¹⁹F-NMR (400 MHz, DMSO-d₆): −97.4 (9F, s)

### Example 11

18 g (87 mmol) of 3,5-di-tert-butylphenol, 6.97 g (87 mmol) of NaOH (a 50% by mass aqueous solution), and water (50 mL) were mixed and stirred at 25° C. for 30 minutes. Then, 38.8 g (113 mmol) of triphenylsulfonium bromide (TPSBr), dichloromethane (200 mL), and water (50 mL) were added thereto and mixed with a 1,000 mL separatory funnel. The organic layer was washed once with 0.01 mol/L HCl (200 mL) and four times with water (200 mL), and the washed organic layer was concentrated to obtain 32.6 g of an intermediate (a compound A-2) (yield: 80%). Subsequently, 4.8 g (10.2 mmol) of the compound A-2 was dissolved in cyclopentyl methyl ether (CPME) (50 mL), and 270 mg of water was further added thereto. Carbon dioxide was blown into the reaction system, and the mixture was stirred at 25° C. for 1 hour and 30 minutes. The generated crystals were filtered and washed twice with CPME (30 mL) to obtain 1.32 g (yield: 40%) of white crystals. As a result of analysis by ¹H-NMR and ¹³C-NMR, it was confirmed that the white crystals were the compound B-1.

### Example 12

The intermediate (the compound A-2) was synthesized by the same method as in Example 11, and in the same manner as in Example 2, the compound A-2 (10.2 mmol) was subsequently reacted with acetic acid (10.2 mmol), and the purification was followed, thereby obtaining an oily product (a compound B-2) at a yield of 75%.

### Example 13

The intermediate (the compound A-2) was synthesized by the same method as in Example 11, and in the same manner as in Example 12, the compound A-2 (10.2 mmol) was subsequently reacted with trifluoroacetic acid (10.2 mmol), and the purification was followed, thereby obtaining an oily product (a compound B-3) at a yield of 70%.

### Example 14

The intermediate (the compound A-2) was synthesized by the same method as in Example 11, and in the same manner as in Example 12, the compound A-2 (10.2 mmol) was subsequently reacted with 3,5-bis(trifluoromethyl)benzoic acid (10.2 mmol), and the purification was followed, thereby obtaining an oily product (a compound B-4) at a yield of 58%.

### Example 15

The intermediate (the compound A-2) was synthesized by the same method as in Example 11, and in the same manner as in Example 12, the compound A-2 (10.2 mmol) was subsequently reacted with pentafluorophenol (10.2 mmol), and the purification wasd followed, thereby obtaining an oily product (the compound B-5) at a yield of 70%.

### Example 16

The intermediate (the compound A-2) was synthesized by the same method as in Example 11, and in the same manner as in Example 12, the compound A-2 (10.2 mmol) was subsequently reacted with bistrifluoroacetamide (10.2 mmol), and the purification was followed, thereby obtaining an oily product (a compound B-6) at a yield of 75%.

### Example 17

The intermediate (the compound A-2) was synthesized by the same method as in Example 11, and in the same manner as in Example 12, the compound A-2 (10.2 mmol) was subsequently reacted with N-(trifluoromethanesulfonyl)trifluoroacetamide (10.2 mmol), and the purification was followed, thereby obtaining an oily product (a compound B-7) at a yield of 65%.

### Example 18

The intermediate (the compound A-2) was synthesized by the same method as in Example 11, and in the same manner as in Example 12, the compound A-2 (10.2 mmol) was subsequently reacted with bistrifluoromethanesulfonylimide (10.2 mmol), and the purification was followed, thereby obtaining an oily product (a compound B-8) at a yield of 50%.

### Example 19

The intermediate (the compound A-2) was synthesized by the same method as in Example 11, and in the same manner as in Example 12, the compound A-2 (10.2 mmol) was subsequently reacted with 2,4,6-triisopropylbenzenesulfonic acid (the compound B-9) (10.2 mmol), and the purification was followed, thereby obtaining an oily product at a yield of 76%.

### Example 20

The intermediate (the compound A-2) was synthesized by the same method as in Example 11, and in the same manner as in Example 12, the compound A-2 (10.2 mmol) was subsequently reacted with nonafluoro-tert-butanol (10.2 mmol), and the purification was followed, thereby obtaining an oily product (a compound B-10) at a yield of 45%.

### Example 21

20 g (87 mmol) of 3,5-bis(trifluoromethyl)phenol (BisCF₃PhOH), 6.97 g (87 mmol) of NaOH (a 50% by mass aqueous solution), and water (50 mL) were mixed and stirred at 25° C. for 30 minutes. Then, 49.0 g (113 mmol) of trimethoxytriphenylsulfonium bromide (OMeTPSBr), dichloromethane (200 mL), and water (50 mL) were added thereto and mixed with a 1,000 mL separatory funnel. The organic layer was washed once with 0.01 mol/L HCl (200 mL) and four times with water (200 mL), and the washed organic layer was concentrated to obtain 48.1 g of an intermediate (a compound A-3) (yield: 95%). Subsequently, 5.9 g (10.2 mmol) of the compound A-3 was dissolved in cyclopentyl methyl ether (CPME) (50 mL), and 270 mg of water was further added thereto. Carbon dioxide was blown into the reaction system, and the mixture was stirred at 25° C. for 1 hour and 30 minutes. The generated crystals were filtered and washed twice with CPME (30 mL) to obtain 2.11 g (yield: 50%) of white crystals. As a result of analysis by ¹H-NMR and ¹³C-NMR, it was confirmed that the white crystals were a hydrogen carbonate triphenylsulfonium salt (a compound B-11).

### Example 22

The intermediate (the compound A-3) was synthesized in the same manner as in Example 21, then 5.0 g (8.58 mmol) of the compound A-3 was dissolved in THF (50 mL), and further, 1.58 g (8.58 mmol) of pentafluorophenol was added thereto, and the mixture was stirred at 25° C. for 3 hours. The reaction solution was concentrated under reduced pressure with a rotary evaporator to obtain 3.5 g (yield: 7S%) of an oily product. As a result of the analysis by ¹H-NMR and ¹⁹F-NMR, it was confirmed that the obtained oily product was a pentafluorophenol tris(3-methoxyphenyl)sulfonium salt (a compound B-12). The results of ¹H-NMR and ¹⁹F-NMR are shown below.

¹H-NMR (400 MHz, DMSO-d₆): 7.89 to 7.76 (15H, m), 3.82 (9H, s),

¹⁹F-NMR (400 MHz, DMSO-d₆): −171.9 (2F, m), −172.2 (2F, m), −196.2 (IF, m)

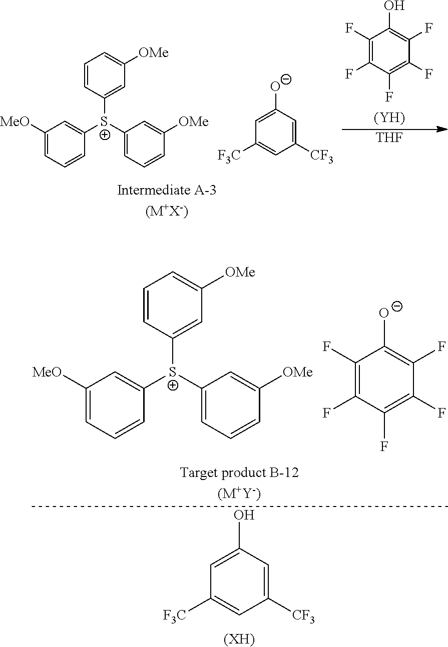

### Example 23

20 g (87 mmol) of 3,5-bis(trifluoromethyl)phenol (BisCF₃PhOH), 6.97 g (87 mmol) of NaOH (a 50% by mass aqueous solution), and water (50 mL) were mixed and stirred at 25° C. for 30 minutes. Thereafter, bis(4-tert-butylphenyl)iodonium bromide (113 mmol), dichloromethane (200 mL), and water (50 mL) were added and mixed with a 1,000 mL separatory funnel. The organic layer was washed once with 0.01 mol/L HCl (200 mL) and four times with water (200 mL), and the washed organic layer was concentrated to obtain an intermediate (a compound A-4) (yield: 70%). Subsequently, in the same manner as in Example 11, the compound A-4 (10.2 mmol) was reacted and purified, thereby obtaining an oily product (a compound B-13) at a yield of 68%.

### Example 24

The intermediate (the compound A-2) was synthesized in the same manner as in Example 11. In the same manner as in Example 2, the compound A-2 (10.2 mmol) was subsequently reacted with tris(trifluoroacetyl)methane (10.2 mmol), and the purification was followed, thereby obtaining a white solid (a compound B-14) at a yield of 95%. The results of ¹H-NMR and ¹⁹F-NMR are shown below.

¹H-NMR (400 MHz, DMSO-d₆): 7.89 to 7.76 (15H, m)

¹⁹F-NMR (400 MHz, DMSO-d₆): −4.1 (9F, s)

### Comparative Example 1

A solution prepared by dissolving 5.11 g (14.9 mmol) of TPSBr in methanol (MeOH) (100 mL) was passed through a strongly basic anion exchange resin in which the anion moiety had been exchanged by a hydrogen carbonate ion. The obtained eluate was concentrated under reduced pressure to distill off MeOH, water was added to the reduced pressure-treated residue and followed by washing with CPME. The aqueous solution was appropriately concentrated to obtain 21.45 g (yield: 88%) of a 20% aqueous solution. Analysis was performed by ¹H-NMR and ¹³ C-NMR, and it was confirmed that the obtained product was the hydrogen carbonate triphenylsulfonium salt (the compound B-1). The results of ¹H-NMR and ¹³C-NMR are shown below.

¹H-NMR (400 MHz, D₂O): 7.77 to 7.60 (15H, m)

¹³C-NMR (400 MHz, D₂O): 124.8, 131.4, 131.9, 135.2, 161.5

### Comparative Example 2

10 g (25.6 mmol) of triphenylsulfonyl iodide (TPSI) was dissolved in MeOH (120 mL), 4.3 g (25.6 mmol) of silver acetate was further added thereto, and the resultant mixture was stirred at 25° C. for 4 hours. The reaction solution was subjected to filtration through Celite, and the reaction solution was concentrated under reduced pressure with a rotary evaporator to obtain 5.8 g (yield: 70%) of an oily product. As a result of analysis by ¹H-NMR, it was confirmed that the obtained oily product was an acetate triphenylsulfonium salt (the compound B-2). The result of ¹H-NMR is shown below.

¹H-NMR (400 MHz, DMSO-d₆): 7.89 to 7.77 (15H, m), 1.54 (3H, s)

### Comparative Example 3

10 g (29.2 mmol) of TPSBr was dissolved in MeOH (120 mL), 7.0 g (30.6 mmol) of silver oxide was further added, and the resultant mixture was stirred at 25° C. for 4 hours. The reaction solution was subjected to filtration through Celite and washed twice with MeOH (60 mL), and further, 5.38 g (29.2 mmol) of pentafluorophenol was added and stirred at 25° C. for 2 hours. The reaction solution was concentrated under reduced pressure with a rotary evaporator, 300 ml of a mixed solvent of methyl-t-butyl ether/hexane= 1/1 was added thereto, and the mixture was stirred at 25° C. for 30 minutes. Then, the supernatant was removed, 250 ml of methyl-t-butyl ether was added thereto so that white crystals were precipitated, and after filtration, the white crystals were washed twice with methyl-t-butyl ether (100 ml) to obtain 9.8 g (yield: 75%) of white crystals. As a result of the analysis by ¹H-NMR and ¹⁹F-NMR, it was confirmed that the obtained white crystals were the pentafluorophenol triphenylsulfonium salt (the compound B-5). The results of ¹H-NMR and ¹⁹F-NMR are shown below.

¹H-NMR (400 MHz, DMSO-d₆): 7.89 to 7.76 (15H, m)

¹⁹F-NMR (400 MHz, DMSO-d₆): −171.9 (2F, m), −172.2 (2F, m), −196.2 (1F, m)

The structures of the intermediates (the compounds A-1 to A-4) and the target products (the compounds B-1 to B-14) synthesized in the above Examples and Comparative Examples are shown below. In the following structures, Me represents a methyl group.

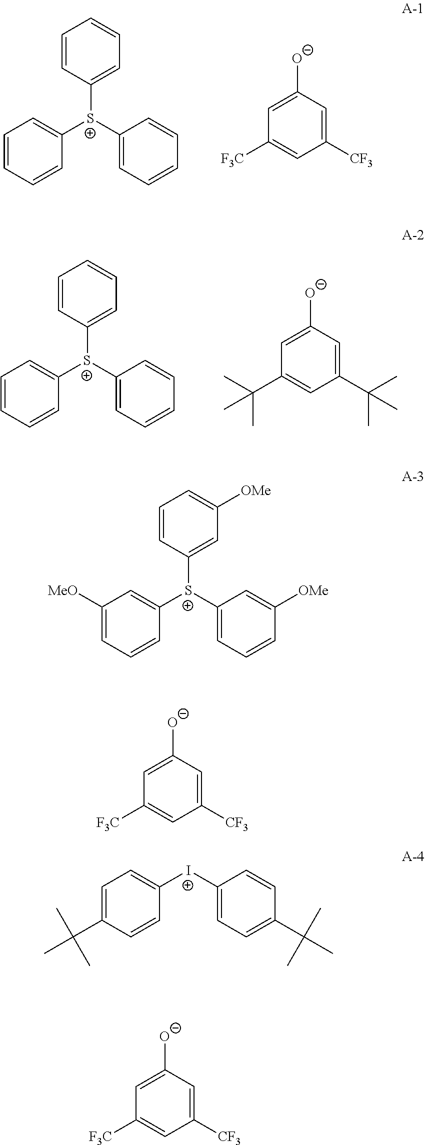

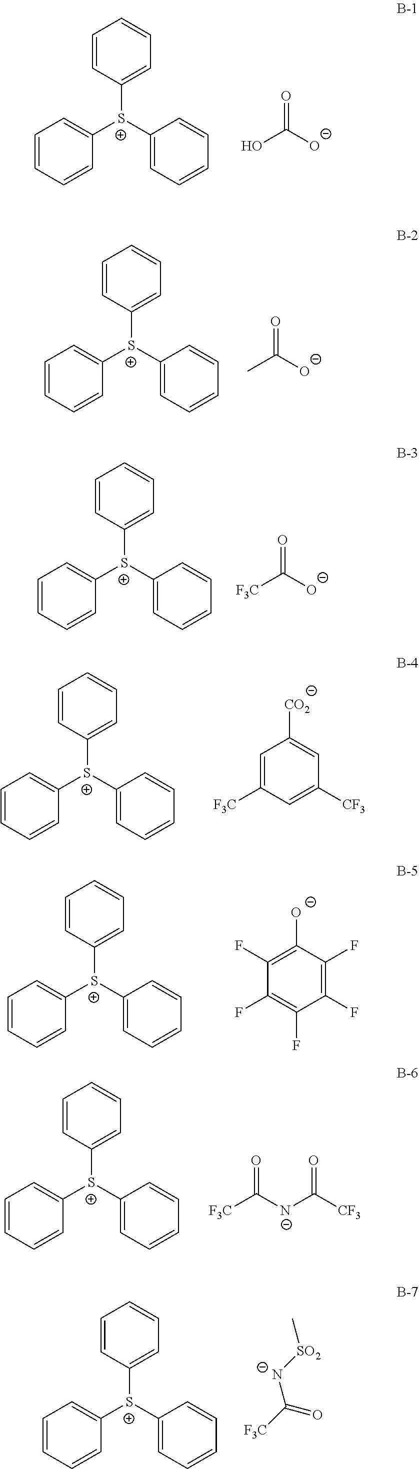

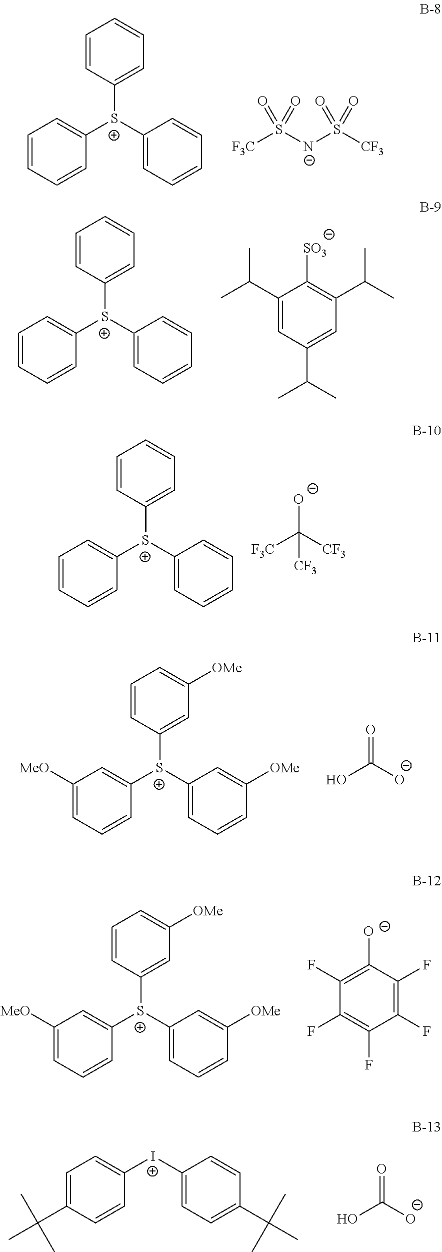

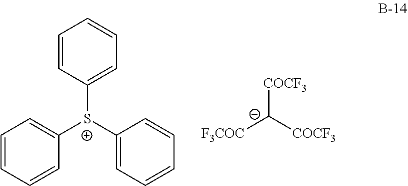

<Evaluation>

The metal contents of the white crystals and the oily products obtained in Examples and Comparative Examples were measured by an ICP emission spectrophotometer. The results are shown in Table 1. It is noted that parts per million (ppm) is based on the mass.

TABLE 1

| | Intermediate (M⁺X⁻) | | | Target product (M⁺Y⁻) | | | Product | | | |
|---|---|---|---|---|---|---|---|---|---|---|
| | | Conjugate acid (XH) | | | Conjugate acid (XH) | | yield | Metal content (ppm) | | |
| | Structure | pKa | ClogP | Structure | pKa | ClogP | (%) | Na | Ca | Ag |
| Example 1 | A-1 | 7.8 | 3.94 | B-1 | 4.4 | −1.75 | 45 | <2* | <2* | <2* |
| Example 2 | A-1 | 7.8 | 3.94 | B-2 | 4.8 | −0.19 | 90 | <2* | <2* | <2* |
| Example 3 | A-1 | 7.8 | 3.94 | B-3 | 0.2 | 0.37 | 75 | <2* | <2* | <2* |
| Example 4 | A-1 | 7.8 | 3.94 | B-4 | 3.3 | 3.88 | 85 | <2* | <2* | <2* |
| Example 5 | A-1 | 7.8 | 3.94 | B-5 | 5.5 | 2.17 | 78 | <2* | <2* | <2* |
| Example 6 | A-1 | 7.8 | 3.94 | B-6 | 3.0 | 0.93 | 90 | <2* | <2* | <2* |
| Example 7 | A-1 | 7.8 | 3.94 | B-7 | −0.4 | 0.77 | 75 | <2* | <2* | <2* |
| Example 8 | A-1 | 7.8 | 3.94 | B-8 | −10.4 | 1.85 | 60 | <2* | <2* | <2* |
| Example 9 | A-1 | 7.8 | 3.94 | B-9 | −0.2 | 3.64 | 82 | <2* | <2* | <2* |
| Example 10 | A-1 | 7.8 | 3.94 | B-10 | 7.1 | 1.77 | 55 | <2* | <2* | <2* |
| Example 11 | A-2 | 10.2 | 5.13 | B-1 | 4.4 | −1.75 | 40 | <2* | <2* | <2* |
| Example 12 | A-2 | 10.2 | 5.13 | B-2 | 4.8 | −0.19 | 75 | <2* | <2* | <2* |
| Example 13 | A-2 | 10.2 | 5.13 | B-3 | 0.2 | 0.37 | 70 | <2* | <2* | <2* |
| Example 14 | A-2 | 10.2 | 5.13 | B-4 | 3.3 | 3.88 | 58 | <2* | <2* | <2* |
| Example 15 | A-2 | 10.2 | 5.13 | B-5 | 5.5 | 2.17 | 70 | <2* | <2* | <2* |
| Example 16 | A-2 | 10.2 | 5.13 | B-6 | 3.0 | 0.93 | 75 | <2* | <2* | <2* |
| Example 17 | A-2 | 10.2 | 5.13 | B-7 | −0.4 | 0.77 | 65 | <2* | <2* | <2* |
| Example 18 | A-2 | 10.2 | 5.13 | B-8 | −10.4 | 1.85 | 50 | <2* | <2* | <2* |
| Example 19 | A-2 | 10.2 | 5.13 | B-9 | −0.2 | 3.64 | 76 | <2* | <2* | <2* |
| Example 20 | A-2 | 10.2 | 5.13 | B-10 | 7.1 | 1.77 | 45 | <2* | <2* | <2* |
| Example 21 | A-3 | 7.8 | 3.94 | B-11 | 4.4 | −1.75 | 75 | <2* | <2* | <2* |
| Example 22 | A-3 | 7.8 | 3.94 | B-12 | 5.5 | 2.17 | 45 | <2* | <2* | <2* |
| Example 23 | A-4 | 7.8 | 3.94 | B-13 | 4.4 | −1.75 | 68 | <2* | <2* | <2* |
| Example 24 | A-2 | 10.2 | 5.13 | B-14 | −2.9 | 0.96 | 90 | <2* | <2* | <2* |
| Comparative | — | — | — | B-1 | 4.4 | −1.75 | 88 | 70 | <2* | <2* |
| Example 1 | | | | | | | | | | |
| Comparative | — | — | — | B-2 | 4.8 | −0.19 | 70 | 120 | 40 | 20 |
| Example 2 | | | | | | | | | | |
| Comparative | — | — | — | B-5 | 5.5 | 2.17 | 75 | 92 | <2* | 4 |
| Example 3 | | | | | | | | | | |

As can be seen from the results in Table 1, in the metal impurities contained in the photo-acid generator obtained by the producing method according to the embodiment of the present invention, all of Na, Ca, and Ag are below the detection limit (less than 2 ppm), and thus the content of metal impurities is significantly low as compared with those of the photo-acid generator of Comparative Example 1, which is produced using the ion exchange resin, and the photo-acid generators of Comparative Examples 2 and 3, which are produced using the silver compound. In Table 1, “*” indicates that it was below the detection limit.

According to the present invention, it is to provide a method of producing a photo-acid generator having a low content of metal impurities.

The present invention has been described in detail and with reference to specific embodiments; however, it is apparent to those skilled in the art that various changes and modifications can be made without departing from the spirit and the scope of the invention.

What is claimed is:

1. A method of producing a salt, comprising:
reacting M⁺X⁻ with YH to generate XH and M⁺Y⁻, and subsequently removing the generated XH to obtain the M⁺Y⁻,
wherein the M⁺X⁻ is a salt of a cation represented by M and an anion represented by X⁻,
the M⁺Y⁻ is a salt of the cation represented by M⁺ and an anion represented by Y⁻,
the XH is a conjugate acid of X⁻,
the YH is a conjugate acid of Y⁻,
the M⁺Y⁻ is a compound that generates an acid upon irradiation with an active ray or a radioactive ray,
a pKa of the XH is larger than a pKa of the YH, and
a ClogP value of XH is larger than 2.

2. The method of producing a salt according to claim 1,
wherein a ClogP value of the YH is smaller than the ClogP value of the XH.

3. The method of producing a salt according to claim 1,
wherein the reaction of the M⁺X⁻ with the YH is carried out in a reaction solvent at −78° C. or higher and 100° C. or lower.

4. The method of producing a salt according to claim 3,
wherein the reaction solvent is an ether-based solvent, an ester-based solvent, a ketone-based solvent, a nitrile-based solvent, an alcohol-based solvent, or a fluorine-based solvent.

5. The method of producing a salt according to claim 1,
wherein a crystal containing the M⁺Y⁻ or an oily product containing the M⁺Y⁻ is obtained by the reaction of the M⁺X⁻ with the YH, and the crystal is washed with a washing solvent or the oily product is distilled off under reduced pressure to remove the XH contained in the crystal or the oily product.

6. The method of producing a salt according to claim 5,
wherein the washing solvent is an ether-based solvent, an ester-based solvent, a ketone-based solvent, a nitrile-based solvent, an alcohol-based solvent, or a fluorine-based solvent.

7. The method of producing a salt according to claim 1,
wherein the M⁺ is a sulfonium ion or an iodonium ion.

8. The method of producing a salt according to claim 1,
wherein the X⁻ and the Y⁻ are each independently an anion represented by any one of General Formulae (1) to (6), 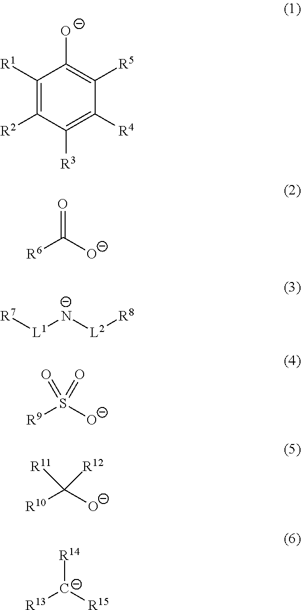
in General Formula (1), R¹ to R⁵ each independently represent a hydrogen atom, a halogen atom, a hydroxy group, or a monovalent organic group, where at least two of R¹, . . . , or R⁵ may be bonded to form a ring structure,
in General Formula (2), R⁶ represents a hydrogen atom, a halogen atom, a hydroxy group, or a monovalent organic group,
in General Formula (3), R⁷ and R⁸ each independently represent a hydrogen atom, a halogen atom, a hydroxy group, or a monovalent organic group, where R⁷ and R⁸ may be bonded to form a ring structure, L¹ represents —SO₂—, —C(═O)—, or a single bond, and L² represents —SO₂— or —C(═O)—,
in General Formula (4), R⁹ represents a hydrogen atom, a halogen atom, a hydroxy group, or a monovalent organic group,
in General Formula (5), R¹⁰ to R¹² each independently represent a hydrogen atom, a halogen atom, a hydroxy group, or a monovalent organic group, and
in General Formula (6), R¹³ to R¹⁵ each independently represent —SO₂—R¹⁶, —C(═O)—R¹⁶, or a cyano group, where R¹⁶ represents a hydrogen atom, a halogen atom, a hydroxy group, or a monovalent organic group.

9. The method of producing a salt according to claim 8,
wherein the X⁻ is an anion represented by General Formula (1) or (3).

10. The method of producing a salt according to claim 8,
wherein the X⁻ is represented by General Formula (1), and at least one of R¹, . . . , or R⁵ in General Formula (1) represents a monovalent organic group.

11. The method of producing a salt according to claim 8,
wherein the Y⁻ is any one of:
an anion represented by General Formula (1), where R¹ to R⁵ in General Formula (1) represent a halogen atom,
an anion represented by General Formula (2), where R⁶ in General Formula (2) represents a hydroxy group or a monovalent organic group,
an anion represented by General Formula (3), where R⁷ and R⁸ in General Formula (3) represent a monovalent organic group,
an anion represented by General Formula (4), where R⁹ in General Formula (4) represents a monovalent organic group,
an anion represented by General Formula (5), where R¹⁰ to R¹² in General Formula (5) each independently represent a monovalent organic group, or
an anion represented by General Formula (6), where R¹³ to R¹⁵ in General Formula (6) each independently represent —SO₂—R¹⁶, where R¹⁶ represents a monovalent organic group.

12. The method of producing a salt according to claim 1, further comprising:
reacting M⁺G⁻ with Q⁺X⁻ to generate the M⁺X⁻ and Q⁺G⁻, and subsequently, removing the generated Q⁺G⁻ to obtain M⁺X⁻.
wherein the G⁻ is a halogen ion, and the Q⁺ is an alkali metal ion or an ammonium ion.

13. The method of producing a salt according to claim 12,
wherein the reaction of the M⁺G⁻ with the Q⁺X⁻ is carried out in a presence of an organic solvent and water, and an obtained organic layer is washed with water to obtain M⁺X⁻.

14. The method of producing a salt according to claim 12,
wherein the G⁻ is a bromine ion or a chlorine ion.
